(19) United States
(12) Patent Application Publication
(10) Pub. No.: US 2012/0178152 A1
(43) Pub. Date: Jul. 12, 2012

(54) MEDICAL HAND-HELD DEVICE

(76) Inventors: Christoph Eisenhardt, Mannheim (DE); Martin Haupt, Wien (AT)

(21) Appl. No.: 13/357,930

(22) Filed: Jan. 25, 2012

Related U.S. Application Data
(63) Continuation of application No. PCT/EP2010/060956, filed on Jul. 28, 2010.

(30) Foreign Application Priority Data
Jul. 30, 2009 (EP) 09166857.4

(51) Int. Cl. C12M 1/34; G01N 21/75

(52) U.S. Cl. 435/288.7; 422/82.05

(57) ABSTRACT

A medical hand-held device is disclosed comprising a test tape unit that can be inserted into a device housing which comprises a test tape provided with a plurality of analytical test elements which can be wound by means of a spool, and a tape drive comprising an electric motor and a gear unit that can be coupled to the test tape unit for winding the test tape forwards in sections, such that the test elements can successively be provided for sample application. The axis of rotation of the electric motor is arranged in a plane extending transversely to the axis of rotation of the spool and that the gear unit has an angle drive to change the direction of the axis.

### CROSS-REFERENCE TO RELATED APPLICATIONS

This application is a continuation of International Application No. PCT/EP2010/060956 filed Jul. 28, 2010, which claims priority to EP Application No. 09166857.4 filed Jul. 30, 2009. Each of the referenced applications is incorporated herein by reference in its entirety.

### FIELD OF THE INVENTION

The invention concerns a medical hand-held device, in particular for blood sugar tests. In one embodiment, the medical hand-held device comprises a test tape unit that can be inserted into a device housing and comprises a test tape provided with a plurality of analytical test elements which can be wound onto a spool, and a tape drive that can be coupled to the test tape unit comprising an electric motor and a gear unit for winding the test tape forwards in sections such that the test elements can successively be provided for sample application, wherein the axis of rotation of the electric motor is arranged in a plane extending transversely to the axis of rotation of the spool.

### BACKGROUND

Test tape instruments are needed that offer additional user advantages compared to the strip systems that are on the market. In practical operation they must ensure that the instrument can be operated discreetly in addition to a reliable positioning of the test elements. Thus, excessive operating noises which can be heard by persons in the vicinity should not occur during use. In order to keep the manufacturing costs, for example, for a blood sugar measuring instrument, within acceptable limits, it is usually necessary to use low-priced components with which it is difficult to adhere to small tolerances. Further requirements are to reduce the volume of the construction and the energy yield of the drive train should be as high as possible.

### SUMMARY

The medical hand-held devices disclosed herein further improve the known systems in the prior art and to achieve a reliable test element positioning with low interfering noises in a compact construction.

According to one aspect, there is provided a medical hand-held device, in particular for blood sugar tests, comprising a test tape unit that can be inserted Into a device housing. The test tape unit comprises a test tape provided with a plurality of analytical test elements, which test tape can be wound by means of a spool, and a tape drive comprising an electric motor and a gear unit that can be coupled to the test tape unit for winding the test tape forwards in sections, such that the test elements can successively be provided for sample application. The axis of rotation of the electric motor is arranged in a plane extending transversely to the axis of rotation of the spool. The electric motor includes a direct current small-size motor supplied by an energy supply of the device and has a rotational speed in the range of 5000 to 15000 revolutions per minute. The gear unit has a ring gear transmission stage with a gear transmission ratio of more than 2 for rotational speed reduction, and the gear unit has an angle drive formed by a crown gear transmission stage for axis direction change, wherein the crown gear transmission stage has a crown gear and a cylindrical gear wheel with straight toothing.

The medical hand-held device disclosed herein is based on the idea of optimizing the arrangement of the drive train especially for tape transport. Accordingly, it is proposed that the axis of rotation of the electric motor is arranged in a plane extending transversely to the axis of rotation of the spool, and that the gear unit has an angle drive for changing the axis direction between the motor axis and the spool axis. This allows a rotary motion to he transferred across axes that are at an angle to one another. This makes it possible to have a very low construction height that is adapted to the characteristics of tape transport whereas at the same time it is possible to utilize more cost-effective drives in the form of conventional small-power motors without giving rise to particularly high demands on mechanical precision. The medical hand-held device disclosed herein includes the angle drive formed by a crown gear transmission stage. In this case power is transmitted by meshing which achieves a substantially higher efficiency compared to a worm drive and moreover, a large reduction in the rotational speed is possible in one transmission stage.

According to one embodiment the gear unit has at least one gear transmission stage formed by a spur-toothed cylindrical gear which is elongated in the direction of the axis of rotation of the electric motor. Thereby, it is possible to separately pre-assemble the different transmission stages on separate carriers (e.g. a bottom of the housing and a separate bearing plate), whereby the cylindrical gear can be paired with a subsequent toothing of lesser tooth width, such that a large axial tolerance is achieved and the final assembly is considerably simplified.

The crown gear transmission stage can have a crown gear and a cylindrical gear wheel with straight toothing. As a result, the axial tolerance is to a large extent uncritical in contrast to bevel gears because the straight-toothed gear wheel can be shifted along its axis without giving rise to problems in the running behaviour. Furthermore, axial forces are not generated even under load.

Another particular embodiment provides that the gear unit has a ring gear transmission stage where the ring gear transmission stage is formed by an internally toothed ring gear and a drive pinion sitting on the motor shaft of the electric motor with its axis within the ring gear. This allows the speed of rotation to be already considerably reduced in the first step so that interfering noises are effectively reduced. Such an internal geared wheel provides the drive pinion encapsulated inside the ring gear, such that a noise minimization is achieved. Further, a reduction of the design dimensions is thereby possible.

In one particular embodiment, the ring gear transmission stage has a gear transmission ratio of more than 2 for rotational speed reduction. In a further refinement of this embodiment, the gear transmission ratio is about 4.

In an additional embodiment, the ring gear can have teeth with concave flanks and the pinion drive can have convexly arched tooth flanks. This results in a larger tooth overlap and thus also in a “softer” noise.

In a further embodiment, the ring gear transmission stage is directly coupled to the electric motor is supported on a bearing on the electric motor. This enables tolerances in the important distance between the motor shaft/pinion and the internal toothing of the first step to be minimized in a cost-effective manner. In this connection the drive pinion and optionally also the bearing for the ring gear can be in the form of metallic components. Due to the relatively high weight of a metallic bearing, a damping of vibrations is achieved, contributing to a noise minimisation.

In another embodiment a rotary speed adaptation with reduced noise behavior is possible due to the fact that the crown gear transmission stage is downstream of the ring gear transmission stage, the motor rotational speed of the electric motor being many times the input rotational speed of the crown gear transmission stage.

In order to be able to reliably transfer the drive forces onto the spoof another embodiment uses a spur gear transmission stage comprising several spur gears which on she output side can be coupled directly with the spool on a drive journal.

Efficient motion transmission is provided in a further embodiment when the spur gears are commonly supported on one side of a bearing plate where the spur gear wheel on the output side directly drives the spool preferably via a spool plate. Due to the common support of the spur gears on one side of the bearing plate, a small tolerance and respectively a high bearing precision can be achieved when assembling the group of components. Moreover, such an arrangement reduces the construction height.

In order to achieve a low-priced and at the same time compact construction, the electric motor can be comprised of a direct current small-size motor supplied by an energy supply on the instrument side and having a rotational speed in the range of 5000 to 15000, preferably of about 9000 revolutions per minute.

Apart from the noise reduction, the hand-held medical device disclosed herein realizes a compact construction. For this purpose in a particular embodiment the electric motor has an elongate motor block and the motor shaft extends in the longitudinal direction of the motor block. In order to further reduce axial play, the motor shaft of the electric motor can be supported in a sintered bearing made of sintered material.

In yet another embodiment, the instrument housing is designed as a test tape unit for exchanging a tape cassette where the maximum thickness of the flat instrument housing is less than 30 mm. In one refinement of this embodiment, the maximum thickness of the flat instrument housing is less than 25 mm.

### BRIEF DESCRIPTION OF THE FIGURES

The invention is further elucidated in the following on the basis of an embodiment example shown in the drawing.

FIG. 1 shows a medical hand-held device with a tape cassette for blood sugar tests in an open perspective diagram.

FIG. 2 shows the tape drive of the hand-held device in an enlargement that is rotated compared to FIG. 1.

FIG. 3 shows the tape drive comprising an electric motor and a gear wheel transmission in a side-view.

FIG. 4 shows two transmission stages of the tape drive in a perspective view.

FIG. 5 shows a sectional enlargement of the ring gear transmission stage according to FIG. 4; and

FIG. 6 shows a diagram of the rotational speed course over the gear train.

### DETAILED DESCRIPTION

The hand-held analyzer shown in FIG. 1 comprises an instrument unit 10 with a particularly smooth-running tape drive 12 and a test tape unit in the form of an exchangeable tape cassette 14 which can be coupled to the tape drive. The instrument allows blood sugar measurements to be carried out locally by the user himself.

The tape cassette 14 shows in FIG. 1 from the underside contains a test tape 16, sections of which are provided with test elements or test fields 18 to the free front side of which blood or body fluid can be applied in the region of a deflection tip at a site of application. At the same time a color change of the respective test field 18 caused by an analyte (blood glucose) can be measured by the optical measuring unit 20 on the rear side at the same position. For this purpose the test tape 16 is pulled out of a store of the cassette 14 from a feed spool by means of the tape drive 12 and re-wound onto a take-up spool 22 such that the spaced apart test fields 18 are successively brought into use at the site of application for successive tests. In this process the take-up spool 22 is rotated around its axis of rotation 25 by the drive journal 24 which engages in a form-locking manner.

The instrument unit 10 has an elongate flat housing 26 the front side of which has a closable loading opening 28 for exchanging the cassette 14. A lancing aid 30 which enables a user to obtain a blood sample by a skin puncture which can then be applied to the test field 18 is flange-mounted on a narrow side of the housing 26. Instrument electronics which are not shown enable the measured values to be processed where the internal energy supply also of the tape drive is by means of batteries 32.

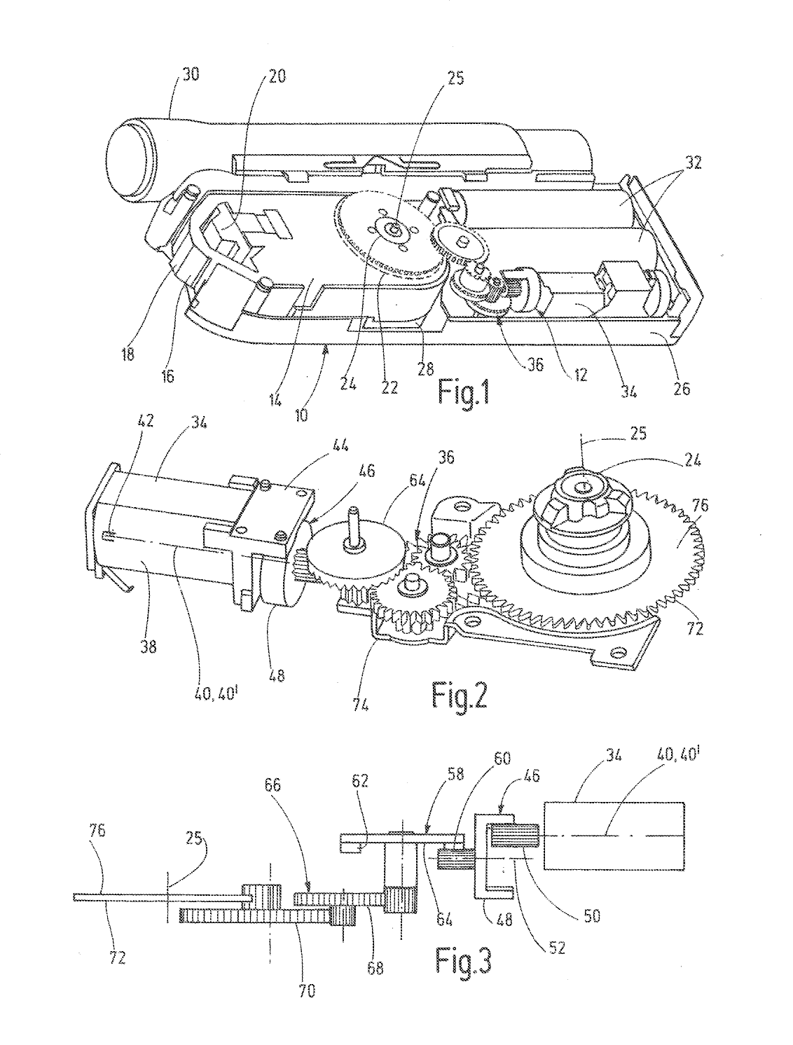

As shown in FIG. 2 the tape drive 12 has an electric motor 34 and a downstream gear unit 36. The electric motor 34 is configured as a direct current small-size motor which can be operated at a high rotational speed of for example up to 10,000 revolutions per minute. It has an elongate motor block 38 and the axis of rotation of the motor 40 and correspondingly of the motor shall 40′ runs in the longitudinal direction of the motor block 38. Due to the horizontal mounting of the motor 34, the axis of rotation of the motor 40 is orientated parallel to a broad side plane of the housing 26 or to the opening face of the loading opening 28 whereas the axis of the spool 25 is perpendicular thereto. In order to reduce axial play, the motor shaft 40′ is situated in sintered bearings 42 (only shown symbolically in FIG. 2). In order to further reduce the tolerance, the bearing block 44 attached to the motor block 38 at the end face is manufactured from a metallic material.

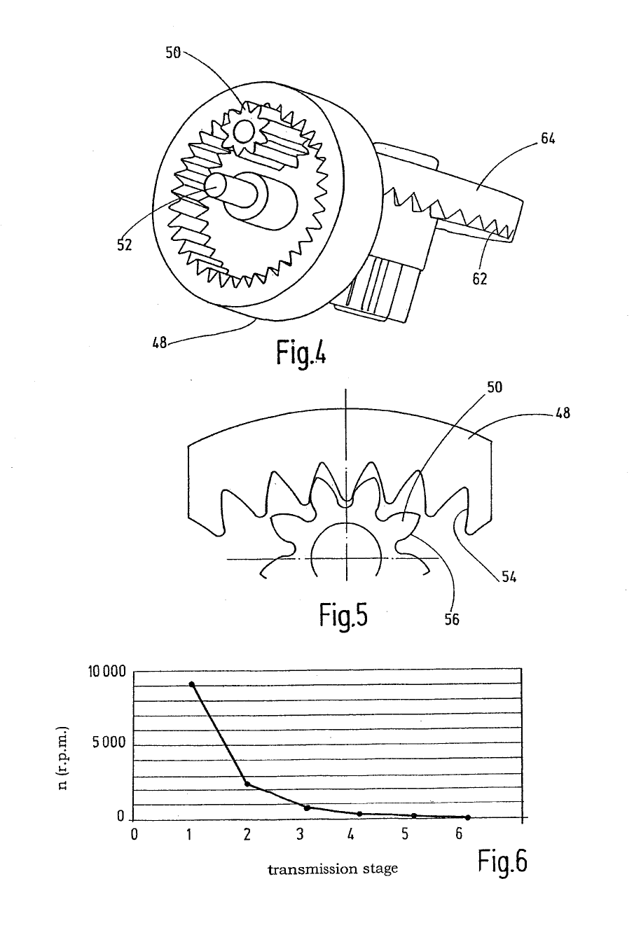

Transfer of the drive movement between the motor 34 and the take-up spool 22 takes place by means of the gear unit 36 which for this purpose is redirected by 90°. In this connection various transmission stages enable a suitable rotational speed and torque adaptation for advancing the test tape 16. The functional arrangement of the gear members can also be seen from the side view of FIG. 3 and the part enlargement of FIG. 4.

The first transmission stage directly coupled to the motor shaft 40′ is in the form of a ring gear transmission stage 46. It comprises an internally toothed ring gear 48 and a drive pinion 50 sitting on the motor shaft 40′ with its axis within the ring gear. The ring gear 48 is advantageously supported on a bearing of the bearing block 44 by means of a bearing pin 52 such that positioning tolerances are substantially minimized and an exact centering of the first transmission stage is ensured.

In order to already considerably reduce the rotational speed and thus the operating noise in the first step, the ring gear transmission stage 46 has a high gear transmission ratio of i=4. In order to achieve a “softer” noise the internal gearing has a large tooth overlap. This can be achieved according to FIG. 5 by providing the teeth of the ring gear 48 with slightly concavely curved tooth flanks 54 whereas the drive pinion 50 has convex or crowned tooth flanks 56.

The subsequent second transmission stage is formed by a crown gear transmission stage 58 which ensures the 90° axis deflection in the drive train. In this case the transmission of motion is by means of a gear wheel connected coaxially with the ring gear 48 where the said gear wheel 60 meshes with the crown toothing 62 of the crown gear 64. The crown gear 64 has an axis which is rotated by 90° relative to the gear wheel 60 and at the same time enables a large reduction of speed in one transmission step. The cylindrical shape of the gear wheel 60 with straight toothing ensures a high axial displacement tolerance without resulting in problems in the running behavior. Low noise and high efficiency are also achieved by a low number of teeth and an optimized tooth geometry of the toothing 62 on the crown gear 64 (see also FIG. 4.).

A third transmission stage is formed by multistage spur gearing 66 which is located downstream of the crown gear 64. This spur gearing comprises several spur gears 68, 70 with axes of rotation that are parallel to one another and which are stably supported expediently together with the crown gear 64 on one side of a common bearing plate 74 (FIG. 2). The gear wheels as injection-moulded parts expediently consist of POM (polyoxymethylene). The last gear wheel 70 is directly connected to a toothed spool plate 72 which is in the form of a stamped metal part the free upper side 76 of which carries the drive pinion 24 for the take-up spool 22.

The spur-toothed cylindrical gears 50, 60 have a tooth width (in axial direction) which is approximately twice the tooth with of the subsequent toothings paired therewith of the ring gear 48 or crown gear 64, so that high axial dislocation tolerance is achieved. In this way, a separate pre-assembly of two transmission stages is possible (drive 34 with ring gear 46 on the body on the one hand, and spur gearing 66 with crown gear 64 on the bearing plate 74 on the other hand), which then can be coupled, without having to impose high demands on axial tolerances during final assembly.

FIG. 6 shows the reduction of rotational speed in the order of the gear members. The motor speed n=9000 rpm is already greatly reduced in the first step by the ring gear transmission stage 46 whereas the subsequent stages result in a more uniform reduction. In the diagram point 1 is assigned to the drive pinion 50, point 2 is assigned to die ring gear 48 and point 3 is assigned to the crown gear 64. The points 4, 5, 6 give the rotational speed of the gear wheels 68, 70 and 72. Thus, on the output side a rotational speed of the drive pinion 24 and thus of die spool 22 of less than 25 revolutions per minute is achieved and the required torque is also available.

Although embodiments of the invention have been described using specific terms, such description is for illustrative purposes only, and it is to be understood that changes and variations obvious to the skilled artisan are to be considered within the scope of the claims that follow and their equivalents.

1. A medical hand-held device in particular for blood sugar tests comprising a test tape unit that can be inserted into a device housing including a test tape provided with a plurality of analytical test elements which test tape can be wound by a spool, and a tape drive comprising an electric motor and a gear unit that can be coupled to the test tape unit for winding the test tape forwards in sections, such that the test elements can successively be provided for sample application, wherein an axis of rotation of the electric motor is arranged in a plane extending transversely to an axis of rotation of the spool, wherein the electric motor comprises a direct current small-size motor supplied by an energy supply of the device and having a rotational speed in the range of 5000 to 15000 revolutions per minute, the gear unit has a ring gear transmission stage with a gear transmission ratio of more than 2 for rotational speed reduction, and the gear unit has an angle drive formed by a crown gear transmission stage for axis direction change, wherein the crown gear transmission stage has a crown gear and a cylindrical gear wheel with straight toothing.

2. The medical hand-held device according to claim 1, wherein at least one of the ring gear transmission stage and the crown gear transmission stage is driven by a spur-toothed cylindrical gear which is elongated in the direction of the axis of rotation of the electric motor.

3. The medical hand-held device according to claim 1, wherein the ring gear transmission stage has a gear transmission ratio of about 4 for rotational speed reduction.

4. The medical hand-held device according to claim 1, wherein the ring gear transmission stage includes an internally toothed ring gear and a drive pinion sitting on a motor shaft of the electric motor with an axis of the drive pinion within the ring gear.

5. The medical hand-held device according to claim 4, wherein the ring gear has teeth with concave flanks and the drive pinion has convexly curved tooth flanks.

6. The medical hand-held device according to claim 4, wherein the ring gear transmission stage is directly coupled to the electric motor and is supported on a bearing on the electric motor.

7. The medical hand-held device according to claim 6, wherein at least one of the drive pinion and the bearing for the ring gear are in the form of metallic components.

8. The medical hand-held device according to claim 1, wherein the crown gear transmission stage is downstream of the ring gear transmission stage, the motor rotational speed of the electric motor being at least two times the input rotational speed of the crown gear transmission stage.

9. The medical hand-held device according to claim 1, further comprising a spar gear transmission stage comprising at least two spur gears having an output side coupled directly to the spool.

10. The medical device of claim 9, wherein the output side of the spur gear transmission stage is coupled to the spool by a drive journal.

11. The medical hand-held device according to claim 9, wherein the spur gears are commonly supported on one side of a bearing plate.

12. The medical hand-held device according to claim 1, wherein the electric motor includes a direct current small-size motor having a rotational speed of about 9000 revolutions per minute.

13. The medical hand-held device according to claim 1, wherein the electric motor has an elongate motor block and a motor shaft that extends in the longitudinal direction of the motor block.

14. The medical hand-held device according to claim 13, wherein the motor shaft of the electric motor is supported in a sintered bearing made of sintered material.

15. The medical hand-held device according to claim 1, wherein the device housing is flat and configured for exchanging a tape cassette as a test tape unit and a maximum thickness of the device housing is less than 30 mm.

16. The medical hand-held device according to claim 15, wherein the maximum thickness of the device housing is less than 25 mm.
